(19) United States
(12) Patent Application Publication
(10) Pub. No.: US 2014/0221270 A1
(43) Pub. Date: Aug. 7, 2014

(54) Methoxypolyethyleneglycol succinimidyl propionate modified recombinant ganoderma immunoregulatory protein, preparing method and application thereof

(71) Applicant: Xitian Zhang, Shanghai (CN)

(72) Inventor: Xitian Zhang, Shanghai (CN)

(21) Appl. No.: 14/119,042

(22) PCT Filed: Jun. 3, 2013
(86) PCT No.: PCT/CN13/76665

§ 371 Date: Nov. 20, 2013

(30) Foreign Application Priority Data
Jul. 16, 2012 (CN) 201210243582.7

(51) Int. Cl. C07K 14/375

(52) U.S. Cl. CPC C07K 14/375
USPC 514/1.1; 530/371

(57) ABSTRACT

Methoxypolyethyleneglycol succinimidyl propionate modified recombinant ganoderma immunoregulatory protein, a preparing method and applications thereof are provided, including: the mPEG-SPA modified rLZ-8; the method for preparing the mPEG-SPA modified rLZ-8 comprising: feeding the rLZ-8 dimer and the mPEG-SPA with the molar ratio of 1:1˜1:6 into a 0.1M phosphate buffer with pH 5.0˜pH 8.0, and stirring by a magnetic stirrer at a room temperature for 1.0˜2.5 h, purifying the product for obtaining a modification product with a purity of 98%; and applications of the mPEG-SPA modified rLZ-8 in preparation of medicine for treating leukopenia due to chemotherapy. Advantages are as follows: the method for preparing the mPEG-SPA modified rLZ-8 is simple, and the product is identical; a half-life of the mPEG-SPA modified rLZ-8 is significantly longer than the half-life of unmodified rLZ-8 (illustrated in the FIG. 2); a minimum effective dosage and time for treating leucopenia are also improved.

### CROSS REFERENCE OF RELATED APPLICATION

This is a U.S. National Stage under 35 U.S.C 371 of the International Application PCT/CN2013/076665, filed Jun. 3, 2013, which claims priority under 35 U.S.C. 119(a-d) to CN 201210243582.7, filed Jul. 16, 2012.

### BACKGROUND OF THE PRESENT INVENTION

1. Field of Invention

The present invention relates to modifying protein with methoxypolyethyleneglycol succinimidyl propionate, and more particularly to methoxypolyethyleneglycol succinimidyl propionate modified recombinant ganoderma immunoregulatory protein rLZ-8 and a preparing method thereof.

2. Description of Related Arts

Recombinant ganoderma immunoregulatory protein (rLZ-8) comes from a mycelium of ganoderma tsugae. A structure of the recombinant ganoderma immunoregulatory protein comprises: an important N-terminal domain for forming a dimer and a C-terminal FNIII domain; wherein the N-terminal domain of the rLZ-8 comprises an α-helix and a β-strand, which forms an important dumbbell-shaped dimer binding domain through space exchanging with the respective domains of another rLZ-8. It has been reported that the rLZ-8 has biological activity on immunoloregulation and killing tumor cells. However, a molecular weight of the dimer is less than 26 kDa; a clearance rate is high; a half-life is short; and pharmacokinetic parameters is difficult to meet requirements of pharmaceutical developments; therefore, only by extending duration of the rLZ-8 in vivo by chemical modification and other technical methods can solid foundations be laid for clinical application.

At present, methoxypolyethyleneglycol (mPEG for short) is usually utilized as a synthesis material for a modifier, a chemical formula is: CH₃O(CH₂CH₂O)nCH₂CH₂OH; one of the terminals is closed by an inert methoxy group in such a manner that molecular crosslinking or agglomeration during modification process can be effectively avoided. As a first generation PEG (polyethyleneglycol)) derivative, PEG-disulfide (PEG-SS for short) has a backbone comprising an ester group which is readily hydrolysable inside a body, and succinate ester fragments left in protein have immunogenicity. As the second generation PEG derivatives, mPEG-succinimidyl propionate (mPEG-SPA for short) and PEG-succinimidyl butyrate (PEG-SBA for short) have no ester group in the backbones in such a manner that a stable connection bond with the protein or polypeptide can be formed, and the mPEG-SPA as well as the PEG-SBA have been widely adapted. However, a modification reaction is a non-directional reaction, and the mPEG-SPA may bind with the respective groups on different sites of the protein; thus, a large number of isomers and by-products will be generated. The half-life of the protein medicine can be extended by being modified with the mPEG, but a characteristic that single-site modification products exist in the modified medicine as well as multi-site modification products is a main technical bottleneck distressing developments of novel medicine quality researches.

Usually, the protein structures need to be preprocessed for obtaining the specific and identical modification products; the readily modified groups on protein chains are substituted or protected, and then the groups are substituted back or unprotected after the modification reaction, but a period of the reaction is extended and a cost is increased, and the biological activity of the protein medicine may be affected. If conditions can be effectively controlled for obtaining identical products with the known structures, safety and controllability of the medicine will be greatly improved and more in line with present guiding principles of novel medicine developments.

### SUMMARY OF THE PRESENT INVENTION

An object of the present invention is to provide an mPEG-SPA modification product of a recombinant ganoderma immunoregulatory protein rLZ-8, a preparation method and applications thereof in preparation of medicine for treating leukopenia due to chemotherapy.

Accordingly, in order to accomplish the above objects, the present invention provides the mPEG-SPA modified rLZ-8, wherein the rLZ-8 dimer is single-site modified at an N-terminal; the rLZ-8 dimer binds with the mPEG-SPA, and a molar ratio thereof is 1:1.

A constitutional formula of the mPEG-SPA is as follows:

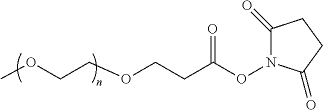

In the constitutional formula, an n value is between 10 and 451, a molecular weight is between 500 Da and 20000 Da.

The present invention also provides the method for preparing the mPEG-SPA modified rLZ-8, comprising steps of:

a) feeding the rLZ-8 dimer and the mPEG-SPA with the molar ratio of 1:1˜1:6 into a 0.1M phosphate buffer with pH 5.0˜pH 8.0, putting in a penicillin bottle, wrapping with tin foil for being away from light, and stirring by a magnetic stirrer at a room temperature for 1˜2.5 h;

b) identifying the product by SDS-PAGE (sodium dodecyl sulfate polyacrylamide gel electrophoresis), staining gel with barium iodide for observing the mPEG-SPA component;

c) purifying and recovering the product, wherein Superdex™75 prep grade chromatography is used for purifying the reaction product, a mobile phase is 0.05M phosphate buffer comprising 0.15M NaCl, the pH is 7.0, a flow rate is 1 mL/min, elution is provided with equivalent concentrations, detection wavelengths are 280 nm, 254 nm, and 215 nm, and the product is collected by a combined method of fixed volume collection and peak collection; and

d) analyzing the purified and recovered samples with the SDS-PAGE, staining the gel with the barium iodide, wherein it is illustrated by identifying with mass spectrometry that the rLZ-8 is only modified at the N-terminal by the PEG, and other sites are not modified; it is further illustrated that the mPEG-SPA modified rLZ-8 obtained in the present invention is the identical modification product; purifying the product for obtaining the modification product with a purity of 98%.

Half-life detections are respectively provided on the modified sample and the original sample, results obtained from an ELISA (enzyme-linked immunosorbent assay) method illustrates that the half-life of the mPEG-SPA modification product is about 2 times of the half-life of the unmodified rLZ-8 dimer.

In the present invention, leukopenia treatments are respectively provided on the modified sample and the original sample, and leukocytes are counted by a cytoanalyzer; the results illustrates that significant difference exits between the rLZ-8 and the mPEG-SPA modification product thereof. Wherein, with a same dosage, a period of the mPEG-SPA modified rLZ-8 for promoting growth of the leukocyte is shorter; with a same treatment period, a number of the leukocyte promoted of the mPEG-SPA modified rLZ-8 is larger. Therefore, it is further illustrated that efficiency of the rLZ-8 on treating the leukopenia is significantly enhanced due to the mPEG-SPA modification.

Therefore, the present invention has advantages as follows: the half-life of the the mPEG-SPA modified rLZ-8 provided by the present invention is significantly extended in comparison to the unmodified rLZ-8; the method for preparing the mPEG-SPA modified rLZ-8 is simple, and the product is identical; under conventional conditions, the mPEG readily modifies on lysine residues, and a primary structure of the rLZ-8 has six lysine residues, that is to say, the rLZ-8 dimer have 12 potential sites for modification, and a large variety of the products with the different modified sites may be produced; the present invention obtains the identical products by controlling the reaction conditions without substituting or protecting any groups as well as other extra process; the method is simple and avoids forming a large variety of the products with the different modified sites; pharmaceutical tests in the present invention proves that the half-life of the mPEG-SPA modified rLZ-8 is significantly longer than the half-life of the unmodified rLZ-8; at the same time, the minimum effective dosage and time for treating leucopenia are also improved.

These and other objectives, features, and advantages of the present invention will become apparent from the following detailed description, the accompanying drawings, and the appended claims.

### BRIEF DESCRIPTION OF THE DRAWINGS

FIG. 1 is electrophoresis results of a purified product after rLZ-8 reacts with mPEG-SPA with a molar ratio of 1:1 according to a preferred embodiment of the present invention.

Wherein: a sample in lane 1 is a protein marker; the sample in lane 2 is the rLZ-8; the sample in lane 3 is the mPEG-SPA; the sample in lane 4 is a mixture after reaction; the sample in lane 5 is a front half portion of a No. 1 collection peak; the sample in lane 6 is a second half portion of the No. 1 collection peak; the sample in lane 7 is a No. 2 collection peak; the sample in lane 8 is a No. 3 collection peak.

FIG. 2 is half-life detection results of mPEG-SPA modified rLZ-8 and original rLZ-8 according to the preferred embodiment of the present invention.

### DETAILED DESCRIPTION OF THE PREFERRED EMBODIMENT

Referring to preferred embodiments, the present invention is further illustrated.

In the following preferred embodiments, rLZ-8 is provided by Jilin University, mPEG-SPA is purchased from Shanghai Yarebio Biological Technology Co., Ltd.

### The Preferred Embodiment 1

### Preparation Reactions and Product Identification of mPEG-SPA Modified rLZ-8

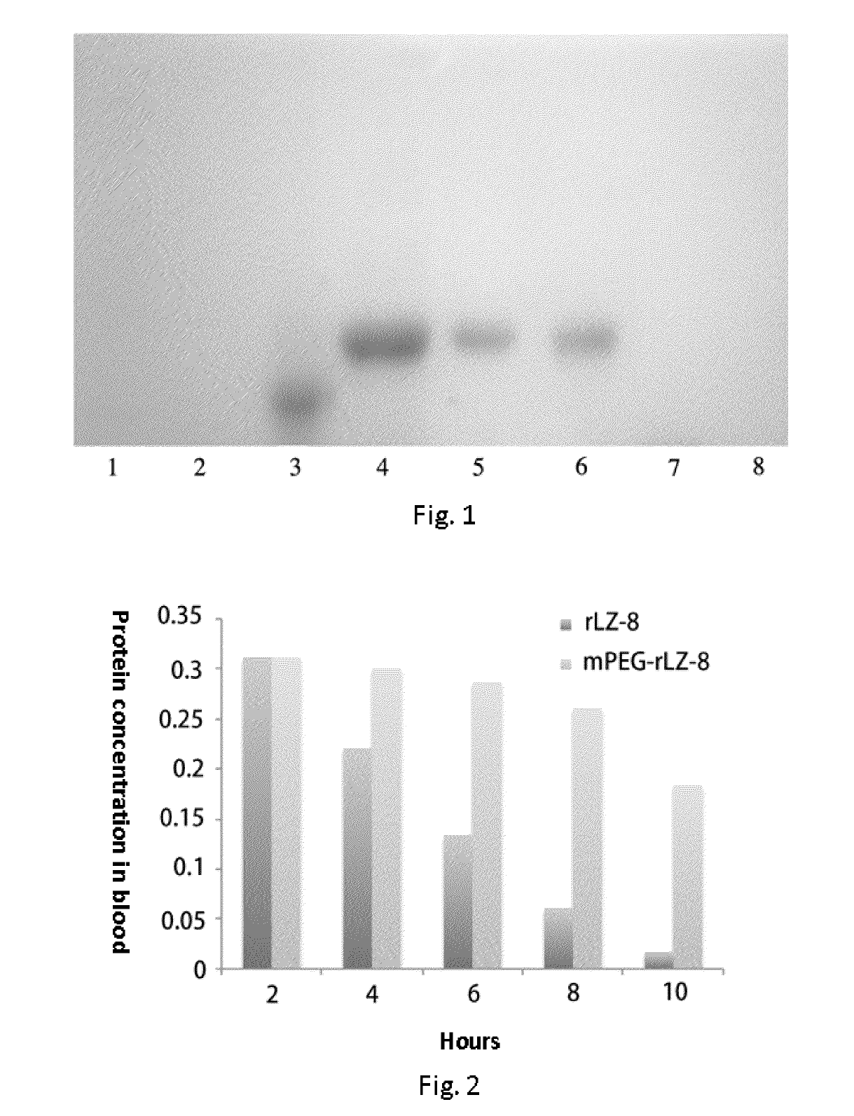

Providing a reaction of the rLZ-8 dimer and the mPEG-SPA (molecular weight is 500 Da) with a molar ratio of 1:1, wherein that is to say, 50 mg the rLZ-8 reacts with 1 mg the mPEG-SPA; wherein a buffer of the reaction is 0.1M phosphate buffer at pH 8.0; putting the mixture in a penicillin bottle, wrapping with tin foil for being away from light, and keeping reacting at a room temperature for 0.5 h; identifying the product by SDS-PAGE, staining gel with barium iodide and analyzing by a gel imaging system, wherein a barium iodide staining technique can provides mPEG-SPA specific staining, and the molecular weight of the mPEG-SPA is relatively small, therefore, when staining the SDS-PAGE gel with the technique (illustrated in the FIG. 1), a band of the mPEG-SPA appears at a low position (illustrated in the FIG. 1, lane 3), and near an edge of a bottom of the gel; however, the relative molecular weight of the modified product increases, the band moves upward (illustrated in the FIG. 1, lane 4), and only one band appears which indicates that components of the product is identical; and

purifying the product with Superdex™75 prep grade chromatography, wherein the purification condition is: a Superdex™75 prep grade (produced by GE Healthcare) chromatogram column (Type Code: XK16/70) is eluted with equivalent concentrations, wherein a mobile phase is 0.05M phosphate buffer comprising 0.15M NaCl, the pH is 7.0, a flow rate is 1 mL/min, detection wavelengths are 280 nm, 254 nm, and 215 nm; collecting the product by a combined method of fixed volume collection and peak collection, obtaining 40 mg the modification product with a purity of 98%.

A mass spectrometry method for identifying modified sites of the mPEG-SPA modified rLZ-8 comprising steps of: providing sample digestion, wherein a sample in a dry powder form is added into 50 mM NH₄HCO₃ and dissolved to the concentration of 1 mg/ml; taking 20 ul sample solution and adding 100 mM DTT (dithiothreitol) to the final concentration of 10 mM, providing the reaction at 56° C. for 1 h; adding 250 mM IAA (indole acetic acid) to the final concentration of 25 mM after cooling to a room temperature, keeping the reaction in dark for 1 h, adding 0.5 ug Trypsin and keeping reaction at 37° C. for 12 h, adding 1 ul 10% TFA (trifluoroacetic acid) and stopping the reaction.

A peptide mass fingerprinting (PMF for short) detection: utilizing a U.S. AB MALDI TOF/TOF™ 5800 mass spectrometer analyzer for detecting the PMF; spotting a mixture of a digested sample and a matrix with a ratio of 1:3 on a target and drying naturally, detecting m/z 500˜m/z 4000 at a positive reflection ion mode, detecting m/z 1000˜m/z 10000 at a positive linear ion mode. A PMF analysis: wherein in the PMF of the modified sample, a matched peptide fragment generally indicates that the peptide is not modified by PEG; sequences of the PEG modified peptide is not matched in the PMF; secondly, the PEG modified site is usually at an N-terminal or a side chain of lysine; furthermore, the PEG modified lysine is generally hard to be digested, therefore, the peptide fragment modified at the lysine by the PEG should comprise at least one omitted site, a molecular weight difference between the PEG modified peptide and the PEG should be approximate to theoretical mass of the peptide fragment, and a mass peak shape of the PEG modified peptide fragment should be basically consistent with the mass peak shape of the PEG.

Results of the detection illustrate that: all the lysine in the PEG modified protein are matched, and it can be judged that the PEG modified sites are not at the lysine; the molecular weight of PEG modified peptide is extremely approximate to the molecular weight of the original PEG, indicating that the PEG modified sites are not at unmatched 75th˜111th amino acid; in the PMF of the PEG modified protein, the fragments with and without methionine are detected at the N-terminals of the protein, which may indicate that some of the PEG modified sites are at the N-terminals of the protein, and during a proteolysis process, some of the methionine are split in such a manner that the peptide fragments without the methionine are matched; in addition, the molecular weight difference between the PEG modified peptide and the original PEG is approximate to the molecular weight of the methionine, which further confirms that the PEG modified sites are at the N-terminals of the protein.

### The Preferred Embodiment 2

### The Preparation Reactions and the Product Identification of the mPEG-SPA Modified rLZ-8

Providing the reaction of the rLZ-8 dimer and the mPEG-SPA (the molecular weight is 5000 Da) with a molar ratio of 1:2 in the 0.1M phosphate buffer at pH 7.0, wherein that is to say, 2.5 mg the rLZ-8 reacts with 1 mg the mPEG-SPA; putting the mixture in the penicillin bottle, wrapping with the tin foil for being away from light, and keeping reacting at the room temperature for 1 h; identifying and purifying the product, wherein the purifying method is the same as in the preferred embodiment 1, obtaining 2.4 mg the modification product with a purity of 98%; wherein the identifying results are the same as in the preferred embodiment 1.

### The Preferred Embodiment 3

### The Preparation Reactions and the Product Identification of the mPEG-SPA Modified rLZ-8

Providing the reaction of the rLZ-8 dimer and the mPEG-SPA (the molecular weight is 10000 Da) with a molar ratio of 1:4 in the 0.1M phosphate buffer at pH 6.0, wherein that is to say, 5 mg the rLZ-8 reacts with 8 mg the mPEG-SPA; putting the mixture in the penicillin bottle, wrapping with the tin foil for being away from light, and keeping reacting at the room temperature for 1.5 h; identifying the product by the SDS-PAGE, staining the gel with the barium iodide and analyzing by the gel imaging system, wherein the identifying and purifying methods are the same as in the preferred embodiment 1, obtaining 5.6 mg the modification product with a purity of 98%; wherein the identifying results are the same as in the preferred embodiment 1.

### The Preferred Embodiment 4

### The Preparation Reactions and the Product Identification of the mPEG-SPA Modified rLZ-8

Providing the reaction of the rLZ-8 dimer and the mPEG-SPA (the molecular weight is 20000 Da) with a molar ratio of 1:6 in the 0.1M phosphate buffer at pH 8.0, wherein that is to say, 5 mg the rLZ-8 reacts with 24 mg the mPEG-SPA; putting the mixture in the penicillin bottle, wrapping with the tin foil for being away from light, and keeping reacting at the room temperature for 2 h; identifying the product by the SDS-PAGE, staining the gel with the barium iodide and analyzing by the gel imaging system, wherein the identifying and purifying methods are the same as in the preferred embodiment 1, obtaining 7.2 mg the modification product with a purity of 98%; wherein the identifying results are the same as in the preferred embodiment 1.

### The Preferred Embodiment 5

### A Half-Life Detection of the mPEG-SPA Modified rLZ-8

Utilizing BALB/c mice weighting about 18˜22 g in experiments, intravenously injecting 100 g/kg the mPEG-SPA (the molecular weight is 10000 Da) modified rLZ-8, respectively sampling blood after 2, 4, 6, 8 and 10 hours after injection, drawing a curve of medicine concentration per time (illustrated in the FIG. 2) with the results obtained, wherein it is indicated by the experiment results that the half-life of the modified protein is significantly extended.

### The Preferred Embodiment 6

### Effects of the mPEG-SPA Modified rLZ-8 on the Leukocytes of Rats

Utilizing Wistar rats in the experiments, wherein 18 rats weighting about 100 g are utilized. A method for preparing reagents comprises steps of: dissolving the rLZ-8 in sterile saline, and diluting into 60 μg/kg, 30 μg/kg and 15 μg/kg dosage groups; dissolving the mPEG-SPA modified rLZ-8 (the molecular weight is 10000 Da) in the sterile saline, and diluting into 60 μg/kg, 30 μg/kg and 15 μg/kg dosage groups; diluting GenLei®Scimax® [recombinant human granulocyte colony-stimulating factor injection (rhG-CSF)], batch number: 20060403, 75 μg/vial, into 13.5 μg/ml and 0.1 ml per rat with the sterile saline; diluting cyclophosphamide (CP) injection, batch number 050216, 200 mg/vial, into 20 mg/ml and 0.1 ml per rat with the sterile saline, or 20 mg/kg.

The experiment has a normal control group, a low-dosage protein group, a middle-dosage protein group, a high-dosage protein group, a low-dosage mPEG-SPA modified rLZ-8 group, a middle-dosage mPEG-SPA modified rLZ-8 group, a high-dosage mPEG-SPA modified rLZ-8 group, and a positive medicine control group (utilizing the GenLei®Scimax®). The rats of each the group are injected with the cyclophosphamide in tail vein for three days except that the sterile saline is given to the normal control group, the dosage is 20 mg/ml and 0.1 ml per rat. On the third day, the blood is sampled from the tail vein, and the leukocytes are counted by a cytoanalyzer. After successful modeling, the rats of each the group are respectively treated with the rLZ-8, the mPEG-SPA modified rLZ-8, or the positive medicine (the GenLei®Scimax®) with the corresponding dosage, and the equal sterile saline is given to the rats of the normal control and a CP group. The blood is sampled from the tail vein on the first, third and seventh treatment days, and the leukocytes are counted by the cytoanalyzer. Medicine efficacy is analyzed according to a number difference of the leukocyte between before and after the treatment.

It is illustrated in Table 1 that on the first treatment day, the leukocyte number of the rats of the mPEG-SPA modified rLZ-8 groups is significantly increased in comparison with the rats of the CP group, and on the seventh treatment day, the leukocyte number basically approaches to a normal level. On the first treatment day, the leukocyte number of the rats of the mPEG-SPA modified rLZ-8 groups is significantly increased in comparison with the rats of the GenLei®Scimax® control group, and on the seventh treatment day, the leukocyte number basically approaches to the normal level. It is emphasized that when compared with the rLZ-8 groups with the same dosage, the mPEG-SPA modification groups have a sufficient leukocyte proliferation effect from the first treatment day, wherein the leukocyte number is about 2 times more than the leukocyte number of the rLZ-8 groups; with a same treatment period, the leukocyte proliferation effect of the low-dosage modified rLZ-8 group is superior to the leukocyte proliferation effect of the high-dosage rLZ-8 group and other rLZ-8 groups.

TABLE 1
effects of rLZ-8 on rats models with leucopenia (x ± s, n = 10)

| | leukocyte | | | |
|---|---|---|---|---|
| | number before | on the first | on the third | on the seventh |
| group | treatment | treatment day | treatment day | treatment day |
| normal control | 14.57 × 10⁹ · L⁻¹ | 13.12 × 10⁹ · L⁻¹ | 12.8 × 10⁹ · L⁻¹ | 9.13 × 10⁹ · L⁻¹ |
| CP control | 4.9 × 10⁹ · L⁻¹ | 5.1 × 10⁹ · L⁻¹ | 5.23 × 10⁹ · L⁻¹ | 10.27 × 10⁹ · L⁻¹ |
| GenLei ® | 4.37 × 10⁹ · L⁻¹ | 5.4 × 10⁹ · L⁻¹ | 10.83 × 10⁹ · L⁻¹* | 10.17 × 10⁹ · L⁻¹ |
| Scimax ® | | | | |
| rLZ-8 (15 μg/kg) | 2.93 × 10⁹ · L⁻¹ | 4.1 × 10⁹ · L⁻¹ | 8.8 × 10⁹ · L⁻¹* | 10.2 × 10⁹ · L⁻¹ |
| rLZ-8 (30 μg/kg) | 2.96 × 10⁹ · L⁻¹ | 4.9 × 10⁹ · L⁻¹ | 9.3 × 10⁹ · L⁻¹* | 12.83 × 10⁹ · L⁻¹ |
| rLZ-8 (60 μg/kg) | 4.33 × 10⁹ · L⁻¹ | 4.4 × 10⁹ · L⁻¹ | 8.6 × 10⁹ · L⁻¹* | 15.5 × 10⁹ · L⁻¹ |
| mPEG-SPA | 3.11 × 10⁹ · L⁻¹ | 8.5 × 10⁹ · L⁻¹* | 10.8 × 10⁹ · L⁻¹ | 13.2 × 10⁹ · L⁻¹ |
| modified rLZ-8 | | | | |
| (10 μg/kg) | | | | |
| mPEG-SPA | 2.89 × 10⁹ · L⁻¹ | 9.2 × 10⁹ · L⁻¹* | 11.98 × 10⁹ · L⁻¹ | 13.4 × 10⁹ · L⁻¹ |
| modified rLZ-8 | | | | |
| (30 μg/kg) | | | | |
| mPEG-SPA | 3.33 × 10⁹ · L⁻¹ | 8.4 × 10⁹ · L⁻¹* | 14.4 × 10⁹ · L⁻¹ | 15.5 × 10⁹ · L⁻¹ |
| modified rLZ-8 | | | | |
| (60 μg/kg) | | | | |

In comparison to CP control group, *p < 0.01

One skilled in the art will understand that the embodiment of the present invention as shown in the drawings and described above is exemplary only and not intended to be limiting.

It will thus be seen that the objects of the present invention have been fully and effectively accomplished. Its embodiments have been shown and described for the purposes of illustrating the functional and structural principles of the present invention and is subject to change without departure from such principles. Therefore, this invention includes all modifications encompassed within the spirit and scope of the following claims.

1-4. (canceled)

5. Methoxypolyethyleneglycol succinimidyl propionate modified recombinant ganoderma immunoregulatory protein rLZ-8, wherein said rLZ-8 dimer is single-site modified at an N-terminal; a molecular ratio of an rLZ-8 dimer molecule and an mPEG-SPA molecule in said mPEG-SPA modified rLZ-8 is 1:1.

6. The mPEG-SPA modified rLZ-8, as recited in claim 1, wherein a constitutional formula of said mPEG-SPA is as follows: 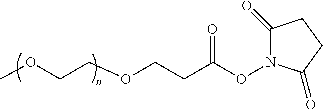
wherein in said constitutional formula, an n value is between 10 and 451, a molecular weight is between 500 Da and 20000 Da.

7. A method for preparing the mPEG-SPA modified rLZ-8, comprising steps of:
a) feeding the rLZ-8 dimer and the mPEG-SPA with the molar ratio of 1:1˜1:6 into a 0.1M phosphate buffer with pH 5.0˜pH 8.0, putting in a penicillin bottle, wrapping with tin foil for being away from light, and stirring by a magnetic stirrer at a room temperature for 1˜2.5 h;
b) identifying the product by SDS-PAGE, staining gel with barium iodide for observing the mPEG-SPA component;
c) purifying and recovering the product, wherein Superdex™75 prep grade chromatography is utilized for purifying the reaction product, a mobile phase is 0.05M phosphate buffer comprising 0.15M NaCl, the pH is 7.0, a flow rate is 1 mL/min, elution is provided with equivalent concentrations, detection wavelengths are 280 nm, 254 nm, and 215 nm, and the product is collected by a combined method of fixed volume collection and peak collection; and
d) analyzing the purified and recovered samples with the SDS-PAGE, staining the gel with the barium iodide, wherein it is illustrated by identifying with mass spectrometry that the rLZ-8 is only modified at the N-terminal by the PEG, and other sites are not modified; it is further illustrated that the mPEG-SPA modified rLZ-8 obtained in the present invention is an identical modification product; purifying the product for obtaining the modification product with a purity of 98%.

8. A method for treating leukopenia due to chemotherapy in a subject, comprising: applying a therapeutically effective amount of the mPEG-SPA modified rLZ-8 as recited in claim 5, or a medicinally acceptable salt thereof, to the subject.
